(19) United States
(12) Patent Application Publication
(10) Pub. No.: US 2024/0321436 A1
(43) Pub. Date: Sep. 26, 2024

(54) EMERGENCY TRANSPORT VEHICLE DISPATCH SYSTEM AND VEHICLE DISPATCH MANAGEMENT DEVICE

(71) Applicant: ALLM INC., Tokyo (JP)

(72) Inventor: Teppei SAKANO, Tokyo (JP)

(21) Appl. No.: 18/579,738

(22) PCT Filed: Aug. 13, 2021
(86) PCT No.: PCT/JP2021/029798

§ 371 Date: Jan. 16, 2024

(51) Int. Cl. G16H 40/20; G16H 10/20

(52) U.S. Cl. CPC G16H 40/20; G16H 10/20

(57) ABSTRACT

An emergency transport dispatch system 10, in which a patient terminal operated by a patient or a person related to the patient, a dispatch terminal operated by a driver of a vehicle that transports the patient, a medical staff terminal operated by a medical worker, and a dispatch management apparatus configured to manage dispatch of vehicles are connected via a communication line to dispatch a vehicle that is not designed specifically for emergency transport for the purpose of emergency transport of the patient.

### TECHNICAL FIELD

The present invention relates to an emergency transport dispatch system and a dispatch management apparatus.

### BACKGROUND ART

The following emergency transport dispatch system is known. In order to dispatch a vehicle that is not designed specifically for emergency transport for the purpose of emergency transport of a patient, the emergency transport dispatch system identifies a disease having a high possibility of occurring in the patient on the basis of triage result information of the patient, decides that a driver who can handle emergency transport of the patient on the basis of the identified disease and has a short travel time to the current location of the patient is a driver to whom a request for transport is made, and transmits request information for requesting the transport of the patient to a dispatch terminal operated by the decided driver (refer to Patent Literature 1).

### CITATION LIST

### Patent Literature

Patent Literature 1: JP-A-2021-86213

### SUMMARY OF INVENTION

### Problems to be Solved by Invention

Patients in need of emergency transport may include those who may be infected with an infectious disease. Hence, when a vehicle that is not designed specifically for emergency transport is dispatched for emergency transport of a patient, if there is a possibility that the patient is infected with an infectious disease, it is preferable to arrange a driver of a vehicle that is appropriate for transport of patients infected with infectious diseases. However, a technology for achieving the above has not been considered at all in the known technology.

### Solution to Problems

According to a first aspect of the present invention, an emergency transport dispatch system, in which a patient terminal operated by a patient or a person related to the patient, a dispatch terminal operated by a driver of a vehicle that transports the patient, a medical staff terminal operated by a medical worker, and a dispatch management apparatus configured to manage dispatch of vehicles are connected via a communication line to dispatch a vehicle that is not designed specifically for emergency transport for the purpose of emergency transport of the patient, the patient terminal includes: a triage result acquisition means configured to acquire a result of triage of the patient; a patient current location identification means configured to identify a current location of the patient; and a triage result information transmission means configured to transmit, to the dispatch management apparatus, triage result information including information on the result of triage of the patient acquired by the triage result acquisition means, information on the current location of the patient identified by the patient current location identification means, and information for verifying an identity of the patient, the dispatch management apparatus includes: a disease identification means configured to identify a disease having a high possibility of occurring in the patient on the basis of the triage result information received from the patient terminal; a driver candidate extraction means configured to perform an extraction process for extracting drivers capable of handling emergency transport of the patient, as driver candidates, from preregistered drivers on the basis of the disease identified by the disease identification means; a driver current location identification means configured to identify a current location of each of the drivers extracted by the driver candidate extraction means; a driver decision means configured to decide that a driver having a short travel time to the current location of the patient among the driver candidates extracted by the driver candidate extraction means is a driver to whom a request for transport is made, on the basis of the current location of the patient and the current locations of the drivers; and a request information transmission means configured to transmit request information for requesting the transport of the patient, the request information including the information indicating the current location of the patient, information indicating the disease having a high possibility of occurring in the patient, and the information for verifying the identity of the patient, to the dispatch terminal operated by the driver decided by the driver decision means, the dispatch terminal includes: a patient information display means configured to display, on a display device, the current location of the patient and the information for verifying the identity of the patient in response to receipt of the request information from the dispatch management apparatus; an arrival detection means configured to detect arrival of the driver at the patient's location; a medical institution extraction means configured to extract medical institutions as candidates for a medical institution to which a request for acceptance of the patient is made, in a case where the arrival detection means has detected the arrival of the driver at the patient's location; a medical institution decision means configured to decide a medical institution having a short travel time among the medical institutions as the candidates for the medical institution to which the request for acceptance is made, which have been extracted by the medical institution extraction means, is the medical institution to which the request for acceptance is made; and a transport information transmission means configured to transmit transport information including an estimated time of arrival at the medical institution, information on a current location of the vehicle, the information indicating the disease having a high possibility of occurring in the patient, and the information for verifying the identity of the patient, to the medical staff terminal operated by a medical worker of the medical institution decided by the medical institution decision means, the medical staff terminal includes a transport information display means configured to display the transport information received from the dispatch terminal on a display device, and in a case where the disease identified by the disease identification means is an infectious disease, the driver candidate extraction means sets an extraction condition in such a manner as to extract drivers of vehicles registered as available for handling transport of infected persons, and performs the extraction process.

According to a second aspect of the present invention, in the emergency transport dispatch system of the first aspect, the triage result acquisition means displays triage questions corresponding respectively to preset triage items on a triage item by triage item basis, obtains responses to the triage questions from an operator, and consequently acquires the result of triage of the patient.

According to a third aspect of the present invention, in the emergency transport dispatch system of the second aspect, the disease identification means presents a possibility of a disease occurring in the patient as a score, for each of preset diseases, on the basis of results of the responses to the triage questions, and identifies a disease having the score exceeding a preset threshold thereof, as the disease having a high possibility of occurring in the patient.

According to a fourth aspect of the present invention, in the emergency transport dispatch system of the third aspect, the driver candidate extraction means sets an extraction condition in such a manner as to extract drivers registered as available for transport for all diseases having the scores exceeding the thresholds thereof, as the driver candidates, and executes the extraction process.

According to a fifth aspect of the present invention, in the emergency transport dispatch system of the fourth aspect, the driver candidate extraction means further sets an extraction condition in such a manner as to extract drivers registered as available for handling unconscious patients in a case where a response to a triage question for checking whether or not the patient is conscious indicates that the patient is unconscious, further sets an extraction condition in such a manner as to extract drivers registered as available for handling patients in cardiopulmonary arrest in a case where a response to a triage question for checking whether or not the patient is in cardiopulmonary arrest indicates that the patient is in cardiopulmonary arrest, and executes the extraction process.

According to a sixth aspect of the present invention, in the emergency transport dispatch system of any of the second to fifth aspects, the triage result acquisition means displays transport target exclusion questions used to determine whether or not to exclude the patient from the transport target, together with the questions corresponding to the triage items, obtains responses to the questions from the operator, and consequently acquires the responses to the transport target exclusion questions, and the emergency transport dispatch system further includes a recommendation means configured to determine that the patient is a patient who is not appropriate for emergency transport using a vehicle that is not designed specifically for emergency transport in a case where the responses to the transport target exclusion questions include a response indicating applicable to the patient, and recommend transport by himself/herself.

According to a seventh aspect of the present invention, in the emergency transport dispatch system of any of the first to sixth aspects, the medical institution extraction means extracts medical institutions capable of handling the disease having a high possibility of occurring in the patient, as the medical institutions as the candidates for the medical institution to which the request for acceptance is made.

According to an eighth aspect of the present invention, in the emergency transport dispatch system of the seventh aspect, in a case where there is a time limit to treatment of the disease having a high possibility of occurring in the patient, the medical institution extraction means narrows down the candidates for the medical institution to which the request for acceptance is made, considering an elapsed time from onset to the present, travel times to the medical institutions, and times from arrival of the patient at the medical institutions to start of treatment.

According to a ninth aspect of the present invention, in the emergency transport dispatch system of any of the first to eighth aspects, in a case where the driver decided by the driver decision means agrees to the transport of the patient, the request information transmission means transmits the request information to the dispatch terminal operated by the driver.

According to a tenth aspect of the present invention, in the emergency transport dispatch system of any of the first to ninth aspects, in a case where the driver decided as the driver to whom the request for transport is made rejects the transport of the patient, the driver decision means decides that a driver having the next shortest travel time to the current location of the patient after the driver who rejected the transport of the patient among the driver candidates extracted by the driver candidate extraction means is the driver to whom the request for transport is made.

According to an eleventh aspect of the present invention, in the emergency transport dispatch system of any of the first to tenth aspects, in a case where the medical worker of the medical institution decided by the medical institution decision means agrees to the acceptance of the patient, the transport information transmission means transmits the transport information to the medical worker terminal operated by the medical worker.

According to a twelfth aspect of the present invention, in the emergency transport dispatch system of any of the first to eleventh aspects, in a case where the medical worker of the medical institution decided as the medical institution to which the request for acceptance is made rejects the acceptance of the patient, the medical institution decision means decides that a medical institution having the next shortest travel time after the medical institution that rejected the acceptance of the patient among the medical institutions as the candidates for the medical institution to which the request for acceptance is made, which have been extracted by the medical institution extraction means, is the medical institution to which the request for acceptance is made.

According to a thirteenth aspect of the present invention, a dispatch management apparatus is a dispatch management apparatus for dispatching a vehicle that is not designed specifically for emergency transport for the purpose of emergency transport of a patient, the dispatch management apparatus including: a triage result acquisition means configured to acquire a result of triage of a patient; a patient current location identification means configured to identify a current location of the patient; a disease identification means configured to identify a disease having a high possibility of occurring in the patient on the basis of the result of triage acquired by the triage result acquisition means; a driver candidate extraction means configured to perform an extraction process for extracting drivers capable of handling emergency transport of the patient, as driver candidates, from preregistered drivers on the basis of the disease identified by the disease identification means; a driver current location identification means configured to identify a current location of each of the drivers extracted by the driver candidate extraction means; a driver decision means configured to decide that a driver having a short travel time to the current location of the patient among the driver candidates extracted by the driver candidate extraction means is a driver to whom a request for transport is made, on the basis of the current location of the patient identified by the patient current location identification means and the current locations of the drivers identified by the driver current location identification means; and a request information transmission means configured to transmit information for requesting the transport of the patient to a terminal operated by the driver decided by the driver decision means, in which in a case where the disease identified by the disease identification means is an infectious disease, the driver candidate extraction means sets an extraction condition in such a manner as to extract drivers of vehicles registered as available for handling transport of infected persons, and performs the extraction process.

According to a fourteenth aspect of the present invention, in the dispatch management apparatus of the thirteenth aspect, the triage result acquisition means displays triage questions corresponding respectively to preset triage items on a triage item by triage item basis, obtains responses to the triage questions from an operator, and consequently acquires the result of triage of the patient.

According to a fifteenth aspect of the present invention, in the dispatch management apparatus of the fourteenth aspect, the disease identification means presents a possibility of a disease occurring in the patient as a score, for each of preset diseases, on the basis of results of the responses to the triage questions, and identifies a disease having the score exceeding a preset threshold thereof, as the disease having a high possibility of occurring in the patient.

According to a sixteenth aspect of the present invention, in the dispatch management apparatus of the fifteenth aspect, the driver candidate extraction means sets an extraction condition in such a manner as to extract drivers registered as available for transport for all diseases having the scores exceeding the thresholds thereof, as the driver candidates, and executes the extraction process.

According to a seventeenth aspect of the present invention, in the dispatch management apparatus of the sixteenth aspect, the driver candidate extraction means further sets an extraction condition in such a manner as to extract drivers registered as available for handling unconscious patients in a case where a response to a triage question for checking whether or not the patient is conscious indicates that the patient is unconscious, further sets an extraction condition in such a manner as to extract drivers registered as available for handling patients in cardiopulmonary arrest in a case where a response to a triage question for checking whether or not the patient is in cardiopulmonary arrest indicates that the patient is in cardiopulmonary arrest, and executes the extraction process.

According to an eighteenth aspect of the present invention, in the dispatch management apparatus of any of the fourteenth to seventeenth aspects, the triage result acquisition means displays transport target exclusion questions used to determine whether or not to exclude the patient from the transport target, together with the questions corresponding to the triage items, obtains responses to the questions from the operator, and consequently acquires the responses to the transport target exclusion questions, and the dispatch management apparatus further includes a recommendation means configured to determine that the patient is a patient who is not appropriate for emergency transport using a vehicle that is not designed specifically for emergency transport in a case where the responses to the transport target exclusion questions include a response indicating applicable to the patient, and recommend transport by himself/herself.

According to a nineteenth aspect of the present invention, in the dispatch management apparatus of any of the thirteenth to the eighteenth aspects, in a case where the driver decided by the driver decision means agrees to the transport of the patient, the request information transmission means transmits the request information to the terminal operated by the driver.

According to a twentieth aspect of the present invention, in the dispatch management apparatus of any of the thirteenth to nineteenth aspects, in a case where the driver decided as the driver to whom the request for transport is made rejects the transport of the patient, the driver decision means decides that a driver having the next shortest travel time to the current location of the patient after the driver who rejected the transport of the patient among the driver candidates extracted by the driver candidate extraction means is the driver to whom the request for transport is made.

### Effects of Invention

According to the present invention, it is configured in such a manner as to identify a disease having a high possibility of occurring in a patient on the basis of the result of triage, set an extraction condition in such a manner as to extract drivers of vehicles registered as available for handling transport of infected persons if the identified disease is an infectious disease, perform an extraction process, and decide on a driver to whom a request for transport is made. Therefore, even if there is a possibility that a patient is infected with an infectious disease, it is possible to dispatch a vehicle of which a driver can handle emergency transport for the purpose of emergency transport of the patient.

### BRIEF DESCRIPTION OF DRAWINGS

FIG. 1 is a block diagram illustrating the configuration of an embodiment of an emergency transport dispatch system 10.

FIG. 2 is a block diagram illustrating the configuration of an embodiment of a patient terminal 100.

FIG. 3 is a block diagram illustrating the configuration of an embodiment of a dispatch terminal 200.

FIG. 4 is a block diagram illustrating the configuration of an embodiment of a medical staff terminal 300.

FIG. 5 is a block diagram illustrating the configuration of an embodiment of a dispatch management apparatus 400.

FIG. 6 is a flowchart diagram illustrating the flow of a process that is executed by the patient terminal 100.

FIG. 7 is a flowchart diagram illustrating the flow of a process that is executed by the dispatch terminal 200.

FIG. 8 is a flowchart diagram illustrating the flow of a process that is executed by the medical staff terminal 300.

FIG. 9 is a flowchart diagram illustrating the flow of a process that is executed by the dispatch management apparatus 400.

### DESCRIPTION OF EMBODIMENTS

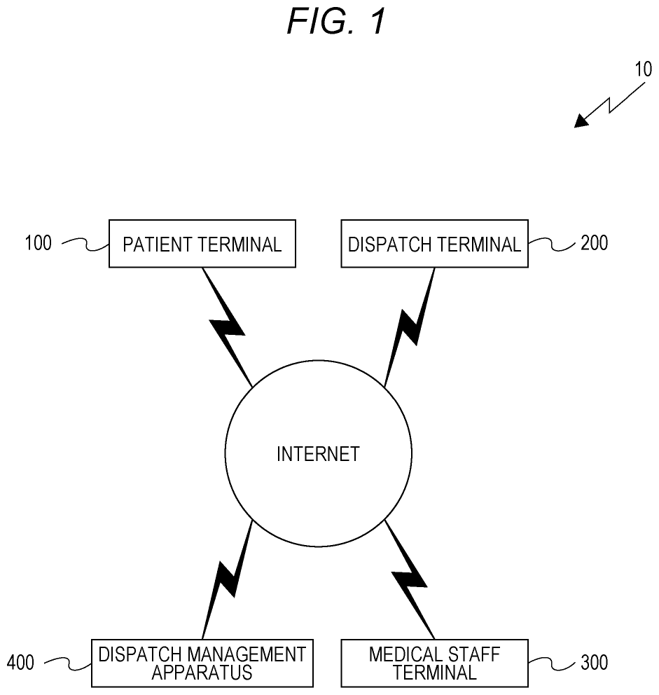

FIG. 1 is a block diagram illustrating the configuration of an embodiment of an emergency transport dispatch system 10 in the embodiment. In the emergency transport dispatch system 10, a patient terminal 100 that is operated by a person concerned such as a patient or a family member of the patient, a dispatch terminal 200 that is operated by a driver of a vehicle, a medical staff terminal 300 that is operated by a medical worker such as a doctor, and a dispatch management apparatus 400 for managing dispatch of vehicles used for emergency transport of patients are connected via a communication line such as the Internet.

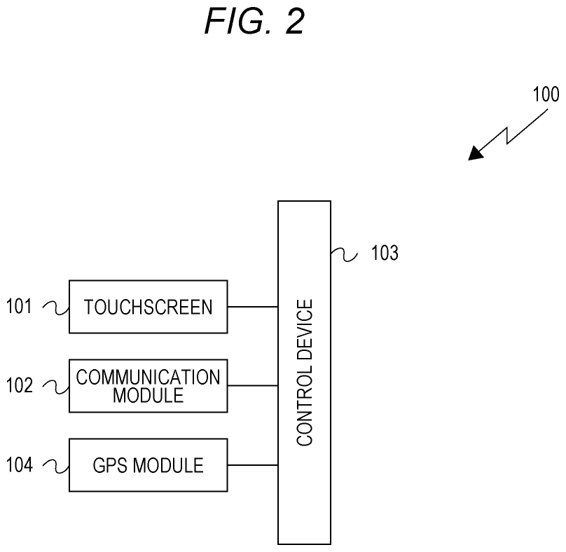

For example, a smartphone, a mobile phone, a tablet terminal, or a personal computer is used as the patient terminal 100. FIG. 2 is a block diagram illustrating the configuration of an embodiment in a case where a smartphone is used as the patient terminal 100 in the embodiment. The patient terminal 100 includes a touchscreen 101, a communication module 102, a control device 103, and a GPS module 104.

The touchscreen 101 is an electronic component in which a display device such as a liquid crystal panel and a position input device such as a touch pad are combined, and is an input device that can operate equipment by pressing a display on the screen. For example, an operator of the patient terminal 100 can operate the patient terminal 100 by touching or sliding a display element such as a button, an icon, or a menu displayed on the liquid crystal panel by use of the finger or a touch pen. The touchscreen 101 detects an operation such as a touch or slide by the operator, and outputs a detection signal for the touch or slide to the control device 103.

A module for connecting to the Internet via a LAN or a mobile phone communication network in a wireless or wired manner is used as the communication module 102.

The control device 103 includes a CPU, memory, and other peripheral circuits, and controls the entire patient terminal 100. Note that the memory configuring the control device 103 includes, for example, volatile memory such as SDRAM and nonvolatile memory such as flash memory. The volatile memory is used as working memory for developing a program when the CPU executes the program, and buffer memory for temporarily recording data. Moreover, program data of firmware for operating the patient terminal 100 and software for operating various applications are recorded in the nonvolatile memory. Note that in the embodiment, program data of a patient application (hereinafter referred to as the “patient app”) described below is recorded in the memory configuring the control device 103.

The GPS module 104 is a module for communicating with GPS satellites and receiving positioning information for identifying the current location from the GPS satellites, and includes an antenna and a control circuit for communicating with the GPS satellites.

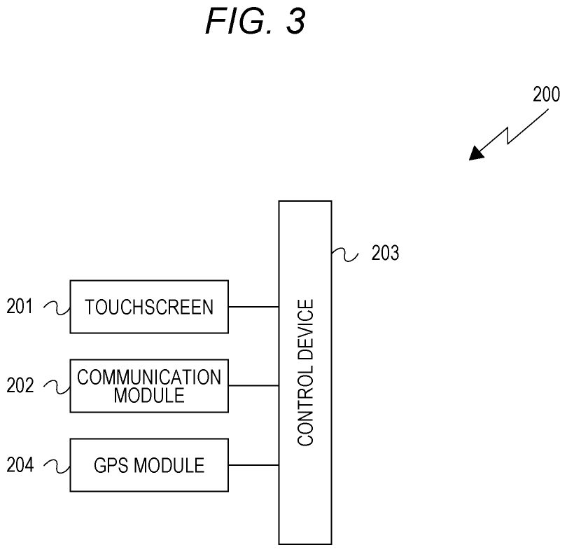

For example, a smartphone, a mobile phone, a tablet terminal, or a personal computer is used as the dispatch terminal 200. A terminal mounted on a vehicle or a terminal held by a driver of a vehicle is assumed as the dispatch terminal 200. FIG. 3 is a block diagram illustrating the configuration of an embodiment in a case where a smartphone is used as the dispatch terminal 200 in the embodiment. The dispatch terminal 200 includes a touchscreen 201, a communication module 202, a control device 203, and a GPS module 204.

The touchscreen 201 is an electronic component in which a display device such as a liquid crystal panel and a position input device such as a touch pad are combined, and is an input device that can operate equipment by pressing a display on the screen. For example, an operator of the dispatch terminal 200 can operate the dispatch terminal 200 by touching or sliding a display element such as a button, an icon, or a menu displayed on the liquid crystal panel by use of the finger or a touch pen. The touchscreen 201 detects an operation such as a touch or slide by the operator, and outputs a detection signal for the touch or slide to the control device 203.

A module for connecting to the Internet via a LAN or a mobile phone communication network in a wireless or wired manner is used as the communication module 202.

The control device 203 includes a CPU, memory, and other peripheral circuits, and controls the entire dispatch terminal 200. Note that the memory configuring the control device 203 includes, for example, volatile memory such as SDRAM and nonvolatile memory such as flash memory. The volatile memory is used as working memory for developing a program when the CPU executes the program, and buffer memory for temporarily recording data. Moreover, program data of firmware for operating the dispatch terminal 200 and software for operating various applications are recorded in the nonvolatile memory. Note that in the embodiment, program data of a dispatch application (hereinafter referred to as the “dispatch app”) described below is recorded in the memory configuring the control device 203.

The GPS module 204 is a module for communicating with GPS satellites and receiving positioning information for identifying the current location from the GPS satellites, and includes an antenna and a control circuit for communicating with the GPS satellites.

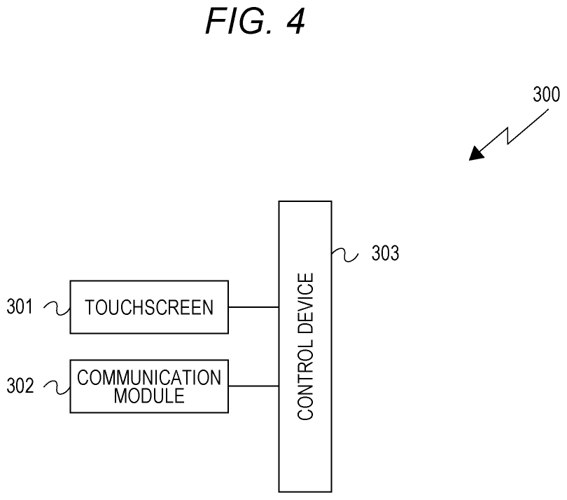

For example, a smartphone, a mobile phone, a tablet terminal, or a personal computer is used as the medical staff terminal 300. FIG. 4 is a block diagram illustrating the configuration of an embodiment in a case where a smartphone is used as the medical staff terminal 300 in the embodiment. The medical staff terminal 300 includes a touchscreen 301, a communication module 302, and a control device 303.

The touchscreen 301 is an electronic component in which a display device such as a liquid crystal panel and a position input device such as a touch pad are combined, and is an input device that can operate equipment by pressing a display on the screen. For example, an operator of the medical staff terminal 300 can operate the medical staff terminal 300 by touching or sliding a display element such as a button, an icon, or a menu displayed on the liquid crystal panel by use of the finger or a touch pen. The touchscreen 301 detects an operation such as a touch or slide by the operator, and outputs a detection signal for the touch or slide to the control device 303.

A module for connecting to the Internet via a LAN or a mobile phone communication network in a wireless or wired manner is used as the communication module 302.

The control device 303 includes a CPU, memory, and other peripheral circuits, and controls the entire medical staff terminal 300. Note that the memory configuring the control device 303 includes, for example, volatile memory such as SDRAM and nonvolatile memory such as flash memory. The volatile memory is used as working memory for developing a program when the CPU executes the program, and buffer memory for temporarily recording data. Moreover, program data of firmware for operating the medical staff terminal 300 and software for operating various applications are recorded in the nonvolatile memory. Note that in the embodiment, program data of a medical staff application (hereinafter referred to as the “medical staff app”) described below is recorded in the memory configuring the control device 303.

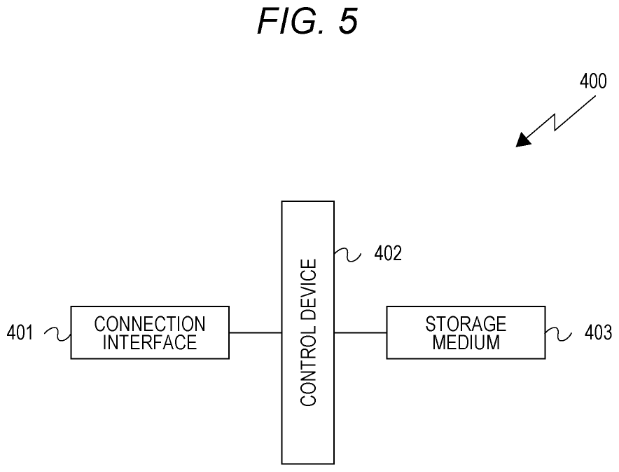

The dispatch management apparatus 400 is an apparatus connected to the Internet. For example, a personal computer or a server is used as the dispatch management apparatus 400. FIG. 5 is a block diagram illustrating the configuration of an embodiment in a case where a server apparatus is used as the dispatch management apparatus 400 in the embodiment. The dispatch management apparatus 400 includes a connection interface 401, a control device 402, and a storage medium 403.

The connection interface 401 is an interface for connecting the dispatch management apparatus 400 to a communication line such as the Internet. Examples of the connection interface 401 include a wired LAN module for wired connection to the Internet, and a wireless LAN module for wireless connection to the Internet.

The control device 402 includes a CPU, memory, and other peripheral circuits, and controls the entire dispatch management apparatus 400. Note that the memory configuring the control device 402 is, for example, volatile memory such as SDRAM. The memory is used as working memory for developing a program when the CPU executes the program, and buffer memory for temporarily recording data. For example, data read via the connection interface 401 is temporarily recorded in the buffer memory.

The storage medium 403 is a storage medium for recording, for example, various kinds of data to be stored in the dispatch management apparatus 400, and data of a program to be executed by the control device 402. For example, an HDD (Hard Disk Drive) or an SSD (Solid State Drive) is used as the storage medium 403. Note that the data of the program to be recorded in the storage medium 403 is provided by being recorded in a recording medium such as a CD-ROM or a DVD-ROM, or is provided via a network, and the data of the program acquired by the operator is installed onto the storage medium 403 to enable the control device 302 to execute the program.

The emergency transport dispatch system 10 in the embodiment is used with the aim of requesting a driver of a vehicle to transport a patient for the purpose of emergency transport of the patient and dispatching the vehicle for the purpose of emergency transport. In the embodiment, the vehicle used for emergency transport is not a vehicle intended for emergency transport such as an ambulance, but is assumed to be a vehicle that is not intended for emergency transport, the vehicle being provided through a service such as a taxi or car sharing. In other words, in the embodiment, a description is given of a method for providing a system for dispatching not a vehicle that is designed specifically for emergency transport, such as an ambulance, but a vehicle that is not designed specifically for emergency transport, such as a taxi, for the purpose of emergency transport of a patient.

There is conventionally, for example, a system in which a taxi can be called using a dispatch app. However, the emergency transport dispatch system 10 in the embodiment provides a mechanism for dispatching a vehicle that is not originally intended for emergency transport, for the purpose of emergency transport of a patient, in cooperation with such an app. As a result, even if it takes time for an ambulance to arrive due to shortage of ambulances, it is possible to swiftly transport a patient to a hospital by making use of a dispatchable vehicle.

In order to use a vehicle that is not intended for emergency transport for the purpose of emergency transport, a driver of the vehicle picks up a patient and transports the patient to a medical institution such as a hospital. Therefore, it is required that the driver and the vehicle can handle emergency transport of the patient. However, a driver of, for example, a taxi does not specialize in emergency transport. Therefore, there is a high possibility that the driver is unaccustomed to handling patients. Moreover, a general vehicle such as a taxi may not have equipment necessary to transport persons infected with infectious diseases, such as equipment for negative pressure. Hence, in the embodiment, a description is given of a method for identifying a driver and a vehicle that can handle emergency transport and targeting the vehicle driven by the driver for dispatch. Details of a process in the emergency transport dispatch system 10 in the embodiment are described below.

When there arises a possibility that a patient needs emergency transport, a person concerned such as the patient or a family member of the patient operates the patient terminal 100 to perform triage of the patient. As described above, the patient app is preinstalled on the patient terminal 100, and an operator such as the patient or the family member can perform triage of the patient by booting the patient app on the patient terminal 100 and instructing start of triage. When the operator instructs startup of the patient app, then the control device 103 reads a program of the patient app from the memory, executes the program, and consequently starts up the patient app.

In the patient app, a plurality of triage items is preset, and the control device 103 displays questions corresponding to the triage items on the touchscreen 101, obtains responses to the questions from the operator, and consequently performs a triage process on the patient. It is assumed that in terms of the content of the question of each of the triage items, contents necessary to perform triage of the patient are preset. In the embodiment, for example, a disease such as intracranial hemorrhage, cerebral infarction, myocardial infarction, or an infectious disease is assumed, and it is assumed that the following triage questions are displayed on the touchscreen 101, and that the operator operates the touchscreen 101 to respond to each of the questions. Note that, in the embodiment, an infectious disease that is determined on whether or not there is a possibility of infection is assumed to be an infectious disease that can be determined on whether or not there is a possibility of infection on the basis of response contents to the triage questions described below.

The operator answers YES or NO to a question: “Does the patient have facial paralysis?” The operator answers YES or NO to a question: “Does the patient have aphasia?” The operator answers YES or NO to a question: “Does the patient have arrhythmia?” The operator answers YES or NO to a question: “Can the patient raise both arms?” The operator answers YES or NO to a question: “Does the patient have dysphonia?” The operator answers YES or NO to a question: “Is the (systolic) blood pressure 165 mmHg or more?” The operator answers YES or NO to a question: “Does the patient have eye deviation?” The operator answers YES or NO to a question: “Does the patient have a headache?” The operator answers YES or NO to a question: “Does the patient's past medical history include cerebral infarction?” The operator answers YES or NO to a question: “Did the patient have chest pain within 24 hours?” The operator answers YES or NO to a question: “Does the patient's past medical history include coronary heart disease?” The operator answers YES or NO to a question: “Does the patient have radiating pain?” The operator answers YES or NO to a question: “Is the patient having a cold sweat?” The operator answers YES or NO to a question: “Does the patient have malaise?” The operator answers YES or NO to a question: “Is SpO2 less than 95%?” The operator answers YES or NO to a question: “Did the patient have close contact with a person infected with an infectious disease within five days?” The operator answers YES or NO to a question: “Is the body temperature 38.5 or more?” The operator answers YES or NO to a question: “Wasn't the patient vaccinated against an epidemic infectious disease 10 or more days ago?” The operator answers the time when the patient developed symptoms, for example, 13:00, to a question: “What time is the onset?” The operator answers YES or NO to a question: “Is the patient conscious?” The operator answers YES or NO to a question: “Is the patient in cardiopulmonary arrest?”

Moreover, transport target exclusion questions used to determine whether or not to exclude the patient from the transport target are prepared in advance, and the control device 103 displays also the following transport target exclusion questions together with the triage questions on the touchscreen 101, and obtains responses from the operator. For example, the following questions are prepared as the transport target exclusion questions.

The operator answers YES or NO to a question: “Is the patient bleeding?” The operator answers YES or NO to a question: “Is the patient behaving violently?” The operator answers YES or NO to a question: “Is the patient rejecting transport to a hospital?”

If judging that the operator has completed responding to the above-mentioned triage questions and transport target exclusion questions, the control device 103 transmits, to the dispatch management apparatus 400, triage result information including information on the triage questions and responses, information on the transport target exclusion questions and responses, information indicating the current location of the patient, information indicating who is not feeling well, the operator himself/herself or a person other than the operator, and information for verifying the identity of the patient.

At this point in time, the control device 103 identifies the current location of the patient terminal 100 on the basis of the output from the GPS module 104, and sets this information as the information indicating the current location of the patient. Moreover, the information indicating who is not feeling well, the operator himself/herself or a person other than the operator, can be obtained by displaying the question for checking whether the person who is not feeling well is the operator himself/herself or a person other than the operator, together with the triage questions and the transport target exclusion questions, and causing the operator to respond to the questions. At this point in time, it may be configured in such a manner that if the person is other than the operator, the person who is not feeling well can be selected from information on preregistered family members, and that information on the selected family member is included in the information indicating who is not feeling well, the operator himself/herself or a person other than the operator. Moreover, it is assumed that the information for verifying the identity of the patient includes data on the patient's name, age, sex, and profile picture, and is inputted and recorded in advance in the memory.

If even one response indicating applicable to the patient, that is, YES, is included in the responses to the transport target exclusion questions, the control device 103 displays information on a nearby medical institution on the touchscreen 101 and recommends transport to the medical institution by himself/herself to the patient or his/her attendant such as a family member of the patient. As a result, in a case of a patient who is not appropriate for transport using a vehicle that is not intended for emergency transport, such as if a patient is behaving violently, bleeding, or rejecting transport, it is possible to recommend transport by himself/herself to the patient or his/her attendant such as a family member of the patient, and the patient.

In the embodiment, for example, latitude and longitude information and address information, which indicate where medical institutions are located, are prerecorded, and the control device 103 is simply required to identify a nearby medical institution on the basis of the location information of the patient terminal 100 acquired from the GPS module 104 and display the nearby medical institution on the touchscreen 101. Note that, at this point in time, information on the nearby medical institution on the touchscreen 101 may be, for example, text information such as the medical institution's name, address, and telephone number, or map information indicating the location of the medical institution on a map.

In the dispatch management apparatus 400, when having received the triage result information from the patient terminal 100, the control apparatus 402 executes a process for deciding on a driver to whom a request for transport of the patient is made on the basis of the received information. The process that is executed by the control device 402 in the dispatch management apparatus 400 is described below.

The control device 402 identifies a disease having a high possibility of occurring in the patient on the basis of the received triage result information. In the embodiment, a description is given of a method for assuming, for example, four diseases—intracranial hemorrhage, cerebral infarction, myocardial infarction, and an infectious diseases—and determining whether or not there is a possibility that any of intracranial hemorrhage, cerebral infarction, myocardial infarction, and an infectious disease is occurring in a patient.

For this purpose, the control device 402 executes the following calculation process on the basis of data on the triage questions and responses to the triage questions, which are included in the triage result information. Note that in the following calculation process, each of an initial value of a score indicating the possibility of intracranial hemorrhage, an initial value of a score indicating the possibility of cerebral infarction, an initial value of a score indicating the possibility of myocardial infarction, and an initial value of a score indicating the possibility of an infectious disease is zero. The control device 402 performs the following score additions or score subtractions on the score indicating the possibility of intracranial hemorrhage, the score indicating the possibility of cerebral infarction, the score indicating the possibility of myocardial infarction, and the score indicating the possibility of an infectious disease, and therefore identifies which disease has a high possibility of occurring in the patient, intracranial hemorrhage, cerebral infarction, or myocardial infarction, or whether or not there is a high possibility that the patient is infected with an infectious disease. Note that examples of the score additions and score subtractions, which are presented below, are merely examples, and the calculation process is not limited to the following examples.

If the response to the triage question: “Does the patient have facial paralysis?” is YES, add two to the score indicating the possibility of intracranial hemorrhage, and add three to the score indicating the possibility of cerebral infarction. If the response to the triage question: “Does the patient have aphasia?” is YES, add one to the score indicating the possibility of intracranial hemorrhage, and add one to the score indicating the possibility of cerebral infarction. If the response to the triage question: “Does the patient have arrhythmia?” is YES, subtract two from the score indicating the possibility of intracranial hemorrhage, add two to the score indicating the possibility of cerebral infarction, and add two points to the score indicating the possibility of myocardial infarction. If the response to the triage question: “Can the patient raise both arms?” is YES, add two to the score indicating the possibility of intracranial hemorrhage, and add three to the score indicating the possibility of cerebral infarction. If the response to the triage question: “Does the patient have dysphonia?” is YES, add one to the score indicating the possibility of intracranial hemorrhage, and add one to the score indicating the possibility of cerebral infarction. If the response to the triage question: “Is the (systolic) blood pressure 165 mmHg or more?” is YES, add one to the score indicating the possibility of intracranial hemorrhage, and add one to the score indicating the possibility of an infectious disease. If the response to the triage question: “Does the patient have eye deviation?” is YES, add one to the score indicating the possibility of intracranial hemorrhage, and add one to the score indicating the possibility of cerebral infarction. If the response to the triage question: “Does the patient have a headache?” is YES, add one to the score indicating the possibility of intracranial hemorrhage, subtract three from the score indicating the possibility of cerebral infarction, and add one to the score indicating the possibility of an infectious disease. If the response to the triage question: “Does the patient's past medical history include cerebral infarction?” is YES, subtract two from the score indicating the possibility of intracranial hemorrhage. If the response to the triage question: “Did the patient have chest pain within 24 hours?” is YES, add two to the score indicating the possibility of myocardial infarction. If the response to the triage question: “Does the patient's past medical history include coronary heart arrest?” is YES, add two to the score indicating the possibility of myocardial infarction. If the response to the triage question: “Does the patient have radiating pain?” is YES, add one to the score indicating the possibility of myocardial infarction. If the response to the triage question: “Is the patient having a cold sweat?” is YES, add one to the score indicating the possibility of myocardial infarction. If the response to the triage question: “Does the patient have malaise?” is YES, add one to the score indicating the possibility of an infectious disease. If the response to the triage question: “Is SpO2 less than 95%?” is YES, add two to the score indicating the possibility of an infectious disease. If the response to the triage question: “Did the patient have close contact with a person infected with an infectious disease within five days?” is YES, add two to the score indicating the possibility of an infectious disease. If the response to the triage question: “Is the body temperature 38.5 degrees or more?” is YES, add one to the score indicating the possibility of an infectious disease. If the response to the triage question: “Wasn't the patient vaccinated against an epidemic infectious disease 10 or more days ago?” is NO, add two to the score indicating the possibility of an infectious disease.

The control device 402 compares the score indicating the possibility of intracranial hemorrhage, the score indicating the possibility of cerebral infarction, the score indicating the possibility of myocardial infarction, and the score indicating the possibility of an infectious disease, which were calculated in the above process, with an intracranial hemorrhage determination threshold, a cerebral infarction determination threshold, a myocardial infarction determination threshold, and an infectious disease determination threshold, which are preset, and identifies a disease having a score greater than its corresponding determination threshold as the disease having a high possibility of occurring in the patient. For example, if the intracranial hemorrhage determination threshold is six, the cerebral infarction determination threshold is five, the myocardial infarction determination threshold is four, the infectious disease determination threshold is six, the calculated score indicating the possibility of intracranial hemorrhage is three, the calculated score indicating the possibility of cerebral infarction is seven, the calculated score indicating the possibility of myocardial infarction is five, and the calculated score indicating the possibility of an infectious disease is eight, then the score indicating the possibility of cerebral infarction, the score indicating the possibility of myocardial infarction, and the score indicating the possibility of an infectious disease are greater than their corresponding determination thresholds. Therefore, it is possible to determine that the patient may have cerebral infarction and myocardial infarction and may be infected with an infectious disease.

The control device 402 identifies a driver to whom the request for transport of the patient is made, on the basis of the identification result of which disease has a high possibility of occurring in the patient, intracranial hemorrhage, cerebral infarction, myocardial infarction, or an infectious disease, and the response results to the triage questions. In the embodiment, it is assumed that information related to drivers to whom the request for emergency transport is made and information related to vehicles are prerecorded in the storage medium 403. As the information related to drivers, for example, information indicating availability for handling emergency transport, information indicating availability for handling each disease, information indicating availability for handling unconscious patients, and information indicating availability for handling patients in cardiopulmonary arrest are registered together with information such as driver names and sex. Moreover, information indicating availability for handling transport of infected persons is registered as the information related to vehicles. In the embodiment, it is assumed that, for example, a negative-pressure vehicle is registered as available for handling transport of infected persons. Note that in the embodiment, the information indicating availability for handling each disease is information indicating availability for handling patients with intracranial hemorrhage, information indicating availability for handling patients with cerebral infarction, and information indicating availability for handling patients with myocardial infarction.

The control device 402 performs a process for deciding on a driver of a vehicle available for handling the transport of the patient among the registered drivers and vehicles. In order to do this, the control device 402 sets extraction conditions for extracting drivers as candidates from the registered drivers. The control device 402 sets extraction conditions in such a manner as to extract drivers registered as available for transport for all diseases of which scores calculated in the above-mentioned process exceed their corresponding thresholds. For example, if the score indicating the possibility of cerebral infarction and the score indicating the possibility of myocardial infarction are greater than their corresponding determination thresholds as described above, an extraction condition is set in such a manner that drivers registered as available for handling both patients in anxiety stop and patients with myocardial infarction are extracted. Moreover, if the score indicating the possibility of an infectious disease, which was calculated in the above-mentioned process, exceeds its corresponding determination threshold, the control device 402 sets an extraction condition in such a manner as to extract drivers of vehicles registered as available for handling transport of infected persons.

Moreover, if the response to the triage question: “Is the patient conscious?” is NO, the control device 402 sets an extraction condition in such a manner as to extract drivers registered as available for handling unconscious patients. Moreover, if the response to the triage question: “Is the patient in cardiopulmonary arrest?” is YES, the control device 402 sets an extraction condition in such a manner as to extract drivers registered as available for handling patients in cardiopulmonary arrest.

The control device 402 extracts drivers who satisfy all the set extraction conditions as driver candidates available for handling transport of the patient. The control device 402 then decides that a driver having the shortest travel time to the current location of the patient among the extracted driver candidates is a driver to whom the request for transport is made. Note that in the embodiment, it is assumed that the control device 402 acquires information indicating the current locations of the drivers from the dispatch terminals 200, and identifies a driver having the shortest travel time between two points, using known map data, on the basis of the information indicating the current location of the patient included in the triage result information, and the information indicating the current locations of the drivers acquired from the dispatch terminals 200. At this point in time, the control device 402 may acquire the information indicating the current locations of the drivers by inquiring the current locations of the dispatch terminals 200 held by the extracted driver candidates, or may be configured to acquire the current locations from the dispatch terminals 200 at predetermined time intervals, targeting all the registered drivers, and use the latest current locations of the drivers. Alternatively, it may be configured in such a manner that the latest current location information is transmitted regularly from the dispatch terminals 200.

The control device 402 transmits request information for requesting the transport of the patient to the dispatch terminal 200 held by the driver decided as the driver to whom the request for transport of the patient is made, and requests emergency transport of the patient. The request information includes, for example, the information indicating the current location of the patient, the information for verifying the identity of the patient, the information indicating who is not feeling well, the operator himself/herself or a person other than the operator, the contents indicating the triage questions and responses, the score indicating the possibility of intracranial hemorrhage, the score indicating the possibility of cerebral infarction, the score indicating the possibility of myocardial infarction, the score indicating the possibility of an infectious disease, and the information indicating diseases exceeding their corresponding determination thresholds.

In the dispatch terminal 200, when having received the request information from the dispatch management apparatus 400, the control device 203 displays the information on the current location of the patient that is where the patient is picked up and the patient information on a screen of the dispatch app displayed on the touchscreen 201. In terms of the information on the current location of the patient, for example, an address indicating the current location of the patient may be displayed, or the current location of the patient may be presented and displayed on a map. Moreover, in terms of the patient information, for example, the information for verifying the identity of the patient included in the request information is simply required to be displayed. Note that the dispatch app may be started in advance on the dispatch terminal 200, or the control device 203 may be configured in such a manner as to start the dispatch app in response to the receipt of the request information from the dispatch management apparatus 400.

The control device 203 accepts an instruction on whether to agree to or reject the transport the patient on the touchscreen 201 to check with the driver for agreement or rejection. If the driver rejects the transport of the patient, the control device 203 transmits information indicating that the driver rejected the transport of the patient, to the dispatch management apparatus 400.

In the dispatch management apparatus 400, if receiving the information indicating that the driver rejected the transport of the patient from the dispatch terminal 200, the control device 402 decides that a driver having the next shortest travel time to the current location of the patient after the driver who rejected the transport of the patient among the driver candidates extracted in the above-mentioned process is the driver to whom the request for transport is made, and transmits the request information to the dispatch terminal 200 held by the decided driver.

If the driver agrees to the transport of the patient, the control device 203 detects arrival of the driver at the patient's location. A process for detecting the arrival of the driver at the patient's location is not particularly limited. However, for example, an input by the driver indicating that the driver has already arrived may be accepted on the touchscreen 201, or it may be configured in such a manner as to detect the arrival of the driver at the patient's location when it is judged that the vehicle has arrived at the current location of the patient included in the request information on the basis of the output from the GPS module 204.

If detecting the arrival of the driver at the patient's location, the control device 203 calculates an elapsed time from the patient's onset to the present and a travel time to a nearby medical institution. The nearby medical institution is simply required to be assumed in advance to be a medical institution located within a predetermined area from the current location of the patient, such as within a radius of several kilometers from the current location of the patient. Moreover, in terms of the elapsed time from the patient's onset to the present, the control device 203 is simply required to identify the onset time on the basis of the response to the triage question: “What time is the onset?”, and calculate the elapsed time from the onset time to the present.

In terms of the travel time to a nearby medical institution, the control device 203 calculates the travel time between two points, using known map data, on the basis of the current location of the vehicle identified on the basis of the output from the GPS module 204 and the location information of the medical institution located in the neighborhood. Note that the location information of the nearby medical institution is assumed to be identified on the basis of preregistered medical institution information. Pieces of information such as location information of medical institutions, availability of treatment of each disease, times from arrival of a patient at the medical institutions to start of treatment, availability of treatment of each disease at the present time, and contact information of each disease are registered as the medical institution information. Moreover, contact information at which it is possible to chat or make a call is registered as the contact information of each disease.

The control device 203 identifies a disease exceeding its corresponding determination threshold on the basis of the request information, and extracts medical institutions that can handle the disease at the present time as candidates for a medical institution to which a request for acceptance is made. Moreover, if there is a time limit to treatment, the control device 203 narrows down the candidates for the medical institution to which the request for acceptance is made, considering the elapsed time from onset to the present, the travel times to the medical institutions, and the times from the arrival of the patient at the medical institutions to the start of treatment. For example, it is assumed that a surgical thrombectomy has a time limit of within eight hours from onset, and a thrombolytic drug has a time limit of 4.5 hours from onset. In this case, if the onset time is eight o'clock, the current time is ten o'clock, the travel time to a hospital is two hours, and the time from arrival at the hospital to start of treatment is one hour, it takes five hours from onset to start of treatment of the patient. Therefore, in a case of a thrombolytic drug with a time limit of 4.5 hours from onset, the hospital becomes inappropriate and is therefore excluded from the candidates for the medical institution to which the request for acceptance is made. However, in terms of a surgical thrombectomy with a time limit of eight hours from onset, the hospital remains appropriate as the candidate for the medical institution to which the request for acceptance is made.

The control device 203 decides that a medical institution having the shortest travel time among the medical institutions as the candidates for the medical institution to which the request for acceptance is made is the medical institution to which the request for acceptance is made, and contacts at contact information of the disease, which has the largest difference between the score and the corresponding threshold, of the medical institution to which the request for acceptance is made, to request acceptance. As described above, contact information at which it is possible to chat or make a call is registered as contact information of each disease in the medical institution information. Therefore, the control device 203 is simply required to contact at the registered contact information. In the embodiment, the dispatch app and the medical staff app are provided with a function of chatting or making a call between the apps, and the driver and the medical institution to which the request for acceptance is made can chat or make a call. Hence, the driver can check acceptance or rejection by a chat or call with a specialist at the contact information of the disease, which has the largest difference between the score and the corresponding threshold, of the medical institution having the shortest travel time.

If the specialist at the contact information does not respond to the contact or rejects the acceptance of the patient, the control device 203 decides that a medical institution having the next shortest travel time after the medical institution that rejected the acceptance among the medical institutions as the candidates for the medical institution to which the request for acceptance is made is the accepting medical institution, and contacts at the contact information of the disease, which has the largest difference between the score and the corresponding threshold, of the medical institution. In this case, the driver can check acceptance or rejection by a chat or call with a specialist at contact information of the disease, which has the largest difference between the score and the corresponding threshold, of the medical institution having the next shortest travel time. For example, the control device 203 is simply required to display a button for checking acceptance or rejection on a screen that is displayed after a call or a chat is finished, and accept an input of agreement or rejection to acceptance.

If the specialist at the contact information agrees to the acceptance of the patient, the control device 203 transmits, to the medical staff terminal 300 of the accepting medical institution, the transport information including an estimated time of arrival at the medical institution, information on the current location of the vehicle, the contents indicating the triage questions and responses, the score indicating the possibility of intracranial hemorrhage, the score indicating the possibility of cerebral infarction, the score indicating the possibility of myocardial infarction, the score indicating the possibility of an infectious disease, the information indicating the disease exceeding its corresponding determination threshold, and the information for verifying the identity of the patient. Moreover, if the accepting medical institution is decided, the driver checks the accepting medical institution on the touchscreen 201 and transports the patient to the accepting medical institution.

In the medical staff terminal 300, the control device 303 displays the transport information received from the dispatch terminal 200 on a screen of the medical staff app displayed on the touchscreen 301 and notifies the doctor who accepts the patient of the transport information. As a result, the specialist of the medical institution that accepts the patient can grasp in advance, for example, the condition of the patient, and the time when the patient arrives, and prepare for treatment.

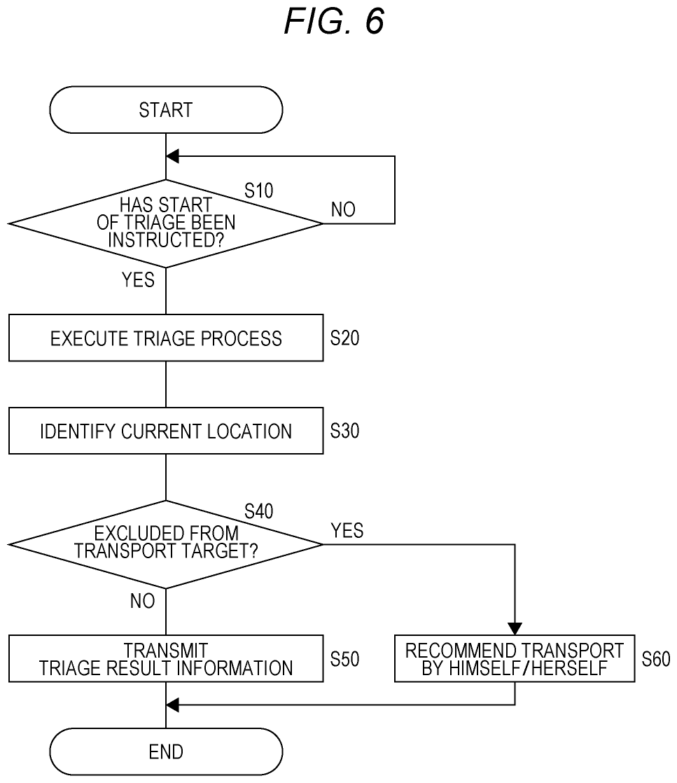

FIG. 6 is a flowchart illustrating the flow of a process that is executed by the patient terminal 100 in the embodiment. The process illustrated in FIG. 6 is executed by the control device 103 as a program that is started when the patient app is executed. Note that execution of the patient app may be instructed by the operator, or may be configured in such a manner as to be automatically instructed when the terminal is switched on.

In step S10, the control device 103 judges whether or not start of triage has been instructed. If an affirmative judgment is made in step S10, the procedure moves on to step S20.

In step S20, as described above, the control device 103 displays the questions corresponding to the triage items on the touchscreen 101, obtains responses to the questions from the operator, and consequently performs the triage process on the patient. The procedure then moves on to step S30.

In step S30, the control device 103 identifies the current location of the patient terminal 100 on the basis of the output from the GPS module 104. The procedure then moves on to step S40.

In step S40, the control device 103 judges whether or not the patient is excluded from the transport target, that is, whether or not even one YES is included in the responses to the transport target exclusion questions. If a negative judgment is made in step S40, the procedure moves on to step S50.

In step S50, the control device 103 transmits the above-mentioned triage result information to the dispatch management apparatus 400. The process then ends.

On the other hand, if an affirmative judgment is made in step S40, the procedure moves on to step S60. In step S60, the control device 103 displays information on a nearby medical institution on the touchscreen 101 and recommends transport to the medical institution by himself/herself to the patient or his/her attendant such as a family member of the patient. The process then ends.

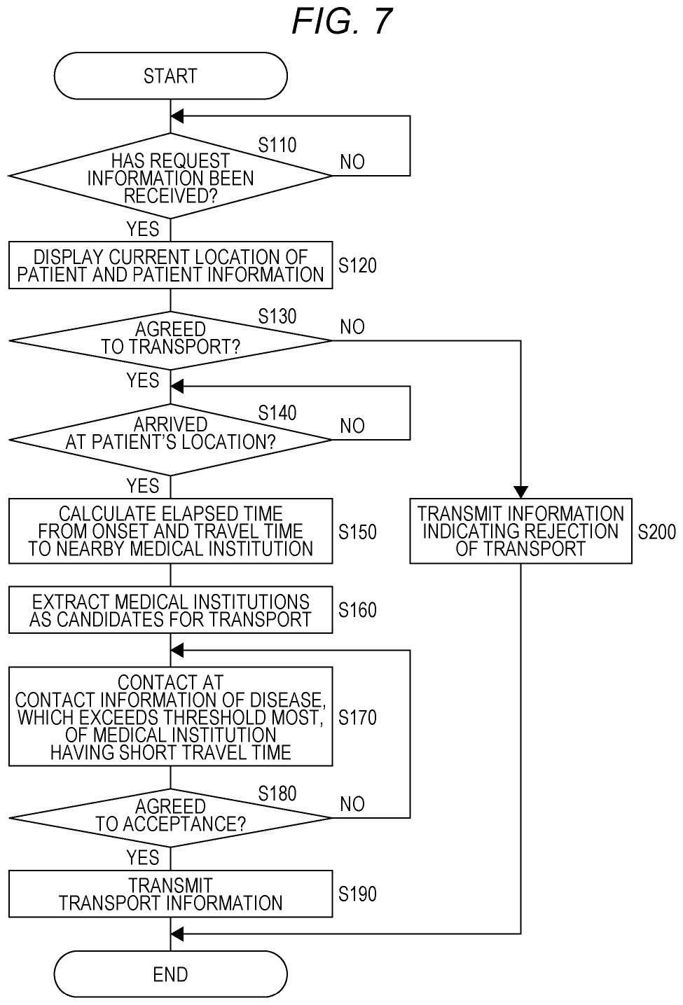

FIG. 7 is a flowchart illustrating the flow of a process that is executed by the dispatch terminal 200 in the embodiment. The process illustrated in FIG. 7 is executed by the control device 203 as a program that is started when the dispatch app is executed. Note that execution of the dispatch app may be instructed by the operator, or may be configured in such a manner as to be automatically instructed when the terminal is switched on.

In step S110, the control device 203 judges whether or not the above-mentioned request information has been received from the dispatch management apparatus 400. If an affirmative judgment is made in step S110, the procedure moves on to step S120.

In step S120, the control device 203 displays the information on the current location of the patient that is where the patient is picked up and the patient information on the screen of the dispatch app displayed on the touchscreen 201. The procedure then moves on to step S130.

In step S130, the control device 203 judges whether or not the driver has agreed to the transport of the patient. If an affirmative judgment is made in step S130, the procedure moves on to step S140.

In step S140, the control device 203 detects whether or not the driver has arrived at the patient's location. If an affirmative judgment is made in step S140, the procedure moves on to step S150.

In step S150, as described above, the control device 203 calculates the elapsed time from the patient's onset to the present and the travel time to a nearby medical institution. The procedure then moves on to step S160.

In step S160, as described above, the control device 203 identifies a disease exceeding its corresponding determination threshold on the basis of the request information, and extracts medical institutions that can handle the disease at the present time as candidates for a medical institution to which a request for acceptance is made. Moreover, if there is a time limit to treatment, the control device 203 narrows down the candidates for the medical institution to which the request for acceptance is made, considering the elapsed time from onset to the present, the travel times to the medical institutions, and the times from arrival of the patient at the medical institutions to start of treatment. The procedure then moves on to step S170.

In step S170, as described above, the control device 203 contacts about the request for acceptance at contact information of the disease, which has the largest difference between the score and the corresponding threshold, of a medical institution having the shortest travel time among the medical institutions as the candidates for the medical institution to which the request for acceptance is made. The procedure then moves on to step S180.

In step S180, the control device 203 judges whether or not a specialist at the contact information has agreed to the acceptance of the patient. If an affirmative judgment is made in step S180, the procedure moves on to step S190.

In step S190, the control device 203 transmits the above-mentioned transport information to the medical staff terminal 300 at the contact information. The process then ends.

On the other hand, if a negative judgment is made in step S180, that is, if the specialist at the contact information does not respond to the contact or rejects the acceptance of the patient, the procedure returns to step S170, and the control device 203 contacts for the request for acceptance at contact information of the disease, which has the largest difference between the score and the corresponding threshold, of a medical institution having the next shortest travel time after the medical institution that rejected the acceptance, and moves on to step S180.

On the other hand, if a negative judgment is made in step S130, that is, if the driver rejects the transport of the patient, the procedure moves on to step S200. In step S200, as described above, the control device 203 transmits information indicating that the driver rejected the transport of the patient to the dispatch management apparatus 400. The process then ends.

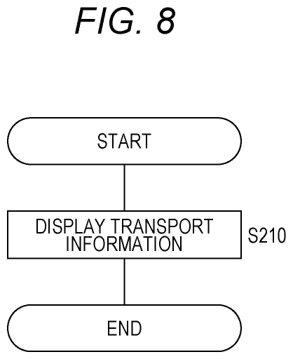

FIG. 8 is a flowchart illustrating the flow of a medical image transmission process that is executed by the medical staff terminal 300 in the embodiment. The process illustrated in FIG. 8 is executed by the control device 303 as a program that is started when the medical staff app is executed. Note that execution of the medical staff app may be instructed by the operator, or may be configured in such a manner as to be automatically instructed when the terminal is switched on.

In step S210, the control device 303 displays the transport information received from the dispatch terminal 200 on the screen of the medical staff app displayed on the touchscreen 301. The process then ends.

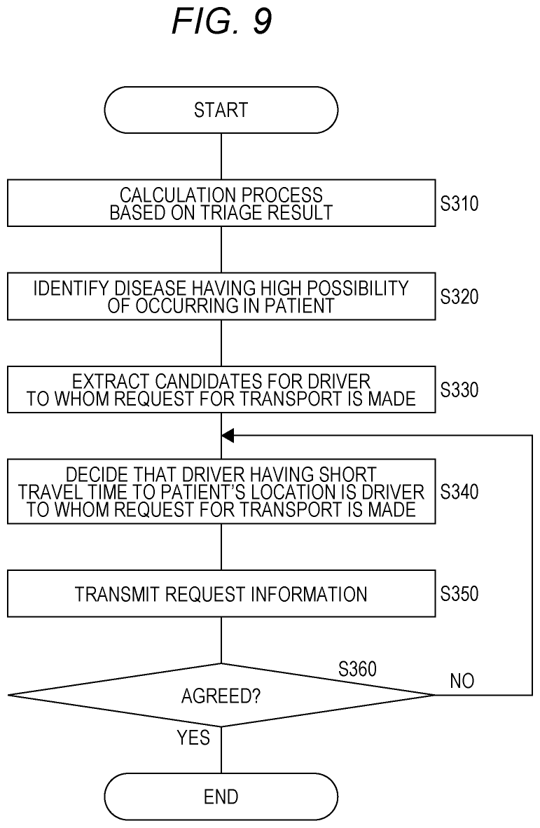

FIG. 9 is a flowchart illustrating the flow of a process that is executed by the dispatch management apparatus 400 in the embodiment. The process illustrated in FIG. 9 is executed by the control device 402 as a program that is started when the above-mentioned triage result information is received from the patient terminal 100.

In step S310, as described above, the control device 402 executes the score calculation process on the basis of the data of the triage questions and the responses to the triage questions, which are included in the triage result information. The procedure then moves on to step S320.

In step S320, as described above, the control device 402 compares a score calculated for each disease with the determination threshold set for each disease, and identifies a disease having a score greater than its corresponding determination threshold as a disease having a high possibility of occurring in the patient. The procedure then moves on to step S330.

In step S330, as described above, the control device 402 extracts candidates for a driver to whom a request for transport of the patient is made, on the basis of the identification result of which disease has a high possibility of occurring in the patient and the response results to the triage questions. The procedure then moves on to step S340.

In step S340, the control device 402 decides that a driver having the shortest travel time to the current location of the patient among the extracted driver candidates is the driver to whom the request for transport is made. The procedure then moves on to step S350.

In step S350, the control device 402 transmits the above-mentioned request information to the dispatch terminal 200 held by the driver decided as the driver to whom the request for the transport of the patient is made. The procedure then moves on to step S360.

In step S360, the control device 402 judges whether or not the driver has agreed to the transport of the patient on the dispatch terminal 200. If an affirmative judgment is made in step S360, the process ends. On the other hand, if a negative judgment is made in step S360, the procedure returns to step S340, and the control device 402 identifies a driver having the next shortest travel time to the patient's location after the driver who rejected the transport, as the driver to whom the request for the transport is made, and moves on to step S350.

According to the embodiment described above, the following operations and effects can be obtained:

(1) In the emergency transport dispatch system 10, the control device 103 of the patient terminal 100 is configured in such a manner as to acquire a result of triage of a patient, identify the current location of the patient, and transmit triage result information including information on the result of triage of the patient, information on the current location of the patient, and information for verifying the identity of the patient, to the dispatch management apparatus 400. The control device 402 of the dispatch management apparatus 400 is configured in such a manner as to identify a disease having a high possibility of occurring in the patient on the basis of the triage result information received from the patient terminal 100, perform the extraction process for extracting, as driver candidates, drivers who can handle emergency transport of the patient from preregistered drivers on the basis of the identified disease, identify the current location of each of the extracted drivers, decide that a driver having a short travel time to the current location of the patient among the driver candidates is a driver to whom a request for transport is made, on the basis of the current location of the patient and the current locations of the drivers, and transmit, to the dispatch terminal 200 operated by the decided driver, request information for requesting the transport of the patient including the information indicating the current location of the patient, information indicating the disease having a high possibility of occurring in the patient, and the information for verifying the identity of the patient. The control device 203 of the dispatch terminal 200 is configured in such a manner as to display the current location of the patient and the information for verifying the identity of the patient on a display device in response to receipt of the request information from the dispatch management apparatus 400, detect arrival of the driver at the patient's location, extract medical institutions as candidates for a medical institution to which a request for acceptance of the patient is made if detecting the arrival of the driver at the patient's location, decide that a medical institution having a short travel time among the extracted medical institutions as the candidates for the medical institution to which the request for acceptance is made is the medical institution to which the request for acceptance is made, and transmit transport information including an estimated time of arrival at the medical institution, information on the current location of the vehicle, the information indicating the disease having a high possibility of occurring in the patient, and the information for verifying the identity of the patient, to the medical staff terminal 300 operated by a medical worker of the decided medical institution. The control device 303 of the medical staff terminal 300 is configured in such a manner as to display the transport information received from the dispatch terminal 200 on a display device. It is configured in such a manner that when drivers are extracted as driver candidates, if the identified disease is an infectious disease, an extraction condition is set in such a manner that drivers of vehicles registered as available for handling transport of infected persons are extracted to perform the extraction process. As a result, it is possible to request a driver of a vehicle that is not designed specifically for emergency transport to transport the patient by making use of a service that provides vehicles that users can use as transportation when necessary, such as taxis, car sharing, and a dispatch app. Hence, even if it takes time for an ambulance to arrive due to shortage of ambulances or insufficient deployment of ambulances, it is possible to dispatch a vehicle for the purpose of emergency transport of a patient and swiftly transport the patient to a hospital. Furthermore, it is configured in such a manner as to identify a disease having a high possibility of occurring in the patient on the basis of the result of triage, and extract drivers who can handle the emergency transport of the patient as driver candidates on the basis of the disease. Therefore, it is possible to dispatch a vehicle of which a driver can handle emergency transport for the purpose of the emergency transport of the patient. Moreover, at an accepting medical institution, a specialist can check in advance information related to the patient to be transported. Furthermore, if there is a possibility that the patient is infected with an infectious disease, it is possible to request emergency transport of the patient to a driver of a vehicle registered as available for handling transport of infected persons.

(2) In the patient terminal 100, the control device 103 is configured in such a manner as to display triage questions corresponding respectively to preset triage items on a triage item by triage item basis, obtain responses to the triage questions from an operator, and consequently acquire the result of triage of the patient. As a result, the operator of the patient terminal 100 can perform triage on the patient by responding to the triage question prepared for each of the triage items.

(3) In the dispatch management apparatus 400, the control apparatus 402 is configured in such a manner as to present a possibility of a disease occurring in the patient as a score, for each of preset diseases, on the basis of results of the responses to the triage questions, and identify a disease having a score exceeding its corresponding preset threshold, as the disease having a high possibility of occurring in the patient. This makes it possible to identify what disease is occurring in the patient on the basis of the result of triage of the patient.

(4) In the dispatch management apparatus 400, the control device 402 is configured in such a manner as to set an extraction condition in such a manner as to extract drivers registered as available for transport for all diseases having scores exceeding their corresponding thresholds, as the driver candidates, and execute the extraction process. As a result, it is possible to extract the drivers who can handle the disease of the patient as the driver candidates.

(5) In the dispatch management apparatus 400, the control device 402 is configured in such a manner as to further set an extraction condition in such a manner as to extract drivers registered as available for handling unconscious patients if the response to the triage question: “Is the patient conscious?” is NO, further set an extraction condition in such a manner as to extract drivers registered as available for handling patients in cardiopulmonary arrest if the response to the triage question: “Is the patient in cardiopulmonary arrest?” is YES, and execute the extraction process. As a result, in the case of unconscious patients and patients in a cardiopulmonary arrest, only drivers who can handle these patients can be extracted as the driver candidates.

(6) In the patient terminal 100, the control device 103 is configured in such a manner as to display the transport target exclusion questions used to determine whether or not to exclude the patient from the transport target, together with the questions corresponding to the triage items, obtain responses to the questions from the operator, and consequently acquire the responses to the transport target exclusion questions, determine that the patient is a patient who is not appropriate for emergency transport using a vehicle that is not designed specifically for emergency transport if the responses to the transport target exclusion questions include a response indicating applicable to the patient, and recommend transport by himself/herself. As a result, it is possible to recommend transport by himself/herself to a patient who is not appropriate for transport using a vehicle that is not intended for emergency transport if it is configured in such a manner as to identify, for example, cases where the patient is behaving violently, bleeding, and rejecting transport by use of the transport target exclusion questions.

(7) In the dispatch terminal 200, the control device 203 is configured in such a manner as to extract medical institutions that can handle the disease having a high possibility of occurring in the patient, as the medical institutions as the candidates for the medical institution to which the request for acceptance is made. As a result, it is possible to extract the medical institutions that can handle treatment of the patient as the candidates for the medical institution to which the request for acceptance is made.

(8) In the dispatch terminal 200, the control device 203 is configured in such a manner as to, if there is a time limit to treatment of the disease having a high possibility of occurring in the patient, narrow down the candidates for the medical institution to which the request for acceptance is made, considering the elapsed time from onset to the present, the travel times to the medical institutions, and the times from arrival of the patient at the medical institutions to start of treatment. As a result, if the total time of the elapsed time from onset to the present, the travel time to a medical institution, and the time from arrival of the patient at the medical institution to start of treatment exceeds the time limit to treatment, it is possible to judge that transport to the medical institution is not possible, exclude the medical institution from the candidates for the medical institution to which the request for acceptance is made, and set, as the candidates for the medical institution to which the request for acceptance is made, only medical institutions of which the total time of the elapsed time from onset to the present, the travel time to a medical institution, and the time from arrival of the patient at the medical institution to start of treatment is within the time limit to treatment.

(9) In the dispatch management apparatus 400, the control device 402 is configured in such a manner as to, if the driver decided as the driver to whom the request for transport is made agrees to the transport of the patient, transmit the request information to the dispatch terminal 200 operated by the driver. As a result, it is possible to transmit the transport information and request the transport of the patient only if the driver is asked in advance about his/her intention and agrees to the emergency transport of the patient.

(10) In the dispatch management apparatus 400, the control device 402 is configured in such a manner as to, if the driver decided as the driver to whom the request for transport is made rejects the transport of the patient, decide that a driver having the next shortest travel time to the current location of the patient after the driver who rejected the transport of the patient among the driver candidates is the driver to whom the request for transport is made. As a result, even if the driver to whom the request for transport is made rejects the transport of the patient, it is possible to request the next nearest driver to transport the patient.

(11) In the dispatch terminal 200, the control device 203 is configured in such a manner as to, if the medical worker of the medical institution decided as the medical institution to which the request for acceptance is made agrees to the acceptance of the patient, transmit the transport information to the medical worker terminal 300 operated by the medical worker. As a result, it is possible to transmit the transport information and request the acceptance of the patient only if the medical worker agrees to acceptance at the medical institution to which the request for acceptance is made.

(12) In the dispatch terminal 200, the control device 203 is configured in such a manner as to, if the medical worker of the medical institution decided as the medical institution to which the request for acceptance is made rejects the acceptance of the patient, decide that a medical institution having the next shortest travel time after the medical institution that rejected the acceptance of the patient among the medical institutions as the candidates for the medical institution to which the request for acceptance is made is the medical institution to which the request for acceptance is made. As a result, even if the medical worker at the medical institution to which the request for acceptance is made rejects the acceptance of the patient, it is possible to request the next nearest medical institution to accept the patient.

### Modifications

Note that it is also possible to modify the emergency transport dispatch system of the above-mentioned embodiment as follows:

(1) In the above-mentioned embodiment, the example is described in which, for example, four diseases—intracranial hemorrhage, cerebral infarction, myocardial infarction, and an infectious disease—are assumed when the control device 402 identifies a disease having a high possibility of occurring in a patient on the basis of triage result information in the dispatch management apparatus 400. However, the target diseases are not limited to these four diseases as long as they are diseases for which the emergency transport dispatch system 10 in the embodiment can be used to decide on a driver to whom a request for transport is made and a medical institution to which a request for acceptance is made, on the basis of the result of triage of the patient, and dispatch a vehicle that is not intended for emergency transport to transport the patient. Moreover, the contents of the questions for triage of the patient may be set in accordance with the assumed diseases.

(2) In the above-mentioned embodiment, the control device 402 of the dispatch management apparatus 400 is configured in such a manner as to further set the extraction condition in such a manner as to extract drivers registered as available for handling unconscious patients if the response to the triage question: “Is the patient conscious?” is NO, further set the extraction condition in such a manner as to extract drivers registered as available for handling patients in cardiopulmonary arrest if the response to the triage question: “Is the patient in cardiopulmonary arrest?” is YES, and execute the extraction process. However, it may be configured in such a manner that other extraction conditions can also be set as long as they are conditions for extracting driver candidates. For example, if the triage result information includes a condition “only female drivers desired,” the control device 402 may, for example, add an extraction condition in such a manner as to extract only drivers whose sex is registered as female.

(3) In the above-mentioned embodiment, the infectious diseases that are determined on whether or not there is a possibility of infection are assumed to be infectious diseases that can be determined on whether or not there is a possibility of infection on the basis of the response contents to the above-mentioned triage questions. However, the target diseases are not limited to these diseases as long as they are diseases for which the emergency transport dispatch system 10 in the embodiment can be used to decide on the driver to whom the request for transport is made and the medical institution to which the request for acceptance is made on the basis of the result of triage of the patient, and dispatch a vehicle that is not intended for emergency transport to transport the patient. Moreover, the contents of the questions for triage of the patient may be set in accordance with the assumed infectious diseases.

Note that the present invention is not at all limited to the configuration in the above-mentioned embodiment as long as the characteristic functions of the present invention are not impaired. Moreover, the configuration may be a combination of the above-mentioned embodiment and a plurality of modifications thereof.

### LIST OF REFERENCE SIGNS

10 Emergency transport dispatch system
100 Patient terminal
101 Touchscreen
102 Communication module
103 Control device
104 GPS module
200 Dispatch terminal
201 Touchscreen
202 Communication module
203 Control device
204 GPS module
300 Medical staff terminal
301 Touchscreen
302 Communication module
303 Control device
400 Dispatch management apparatus
401 Connection interface
402 Control device
403 Storage medium

1. An emergency transport dispatch system for dispatching a vehicle that is not designed specifically for emergency transport for the purpose of emergency transport of a patient, wherein
a patient terminal operated by a patient or a person related to the patient, a dispatch terminal operated by a driver of a vehicle that transports the patient, a medical staff terminal operated by a medical worker, and a dispatch management apparatus configured to manage dispatch of vehicles are connected via a communication line,
the patient terminal includes:
a triage result acquisition means configured to acquire a result of triage of the patient;
a patient current location identification means configured to identify a current location of the patient; and
a triage result information transmission means configured to transmit, to the dispatch management apparatus, triage result information including information on the result of triage of the patient acquired by the triage result acquisition means, information on the current location of the patient identified by the patient current location identification means, and information for verifying an identity of the patient,
the dispatch management apparatus includes:
a disease identification means configured to identify a disease having a high possibility of occurring in the patient on the basis of the triage result information received from the patient terminal;
a driver candidate extraction means configured to perform an extraction process for extracting drivers capable of handling emergency transport of the patient, as driver candidates, from preregistered drivers on the basis of the disease identified by the disease identification means;
a driver current location identification means configured to identify a current location of each of the drivers extracted by the driver candidate extraction means;
a driver decision means configured to decide that a driver having a short travel time to the current location of the patient among the driver candidates extracted by the driver candidate extraction means is a driver to whom a request for transport is made, on the basis of the current location of the patient and the current locations of the drivers; and
a request information transmission means configured to transmit request information for requesting the transport of the patient, the request information including the information indicating the current location of the patient, information indicating the disease having a high possibility of occurring in the patient, and the information for verifying the identity of the patient, to the dispatch terminal operated by the driver decided by the driver decision means,
the dispatch terminal includes:
a patient information display means configured to display, on a display device, the current location of the patient and the information for verifying the identity of the patient in response to receipt of the request information from the dispatch management apparatus;
an arrival detection means configured to detect arrival of the driver at the patient's location;
a medical institution extraction means configured to extract medical institutions as candidates for a medical institution to which a request for acceptance of the patient is made, in a case where the arrival detection means has detected the arrival of the driver at the patient's location;
a medical institution decision means configured to decide that a medical institution having a short travel time among the medical institutions as the candidates for the medical institution to which the request for acceptance is made, which have been extracted by the medical institution extraction means, is the medical institution to which the request for acceptance is made; and
a transport information transmission means configured to transmit transport information including an estimated time of arrival at the medical institution, information on a current location of the vehicle, the information indicating the disease having a high possibility of occurring in the patient, and the information for verifying the identity of the patient, to the medical staff terminal operated by a medical worker of the medical institution decided by the medical institution decision means,
the medical staff terminal includes a transport information display means configured to display the transport information received from the dispatch terminal on a display device, and
in a case where the disease identified by the disease identification means is an infectious disease, the driver candidate extraction means sets an extraction condition in such a manner as to extract drivers of vehicles registered as available for handling transport of infected persons, and performs the extraction process.

2. The emergency transport dispatch system according to claim 1, wherein the triage result acquisition means displays triage questions corresponding respectively to preset triage items on a triage item by triage item basis, obtains responses to the triage questions from an operator, and consequently acquires the result of triage of the patient.

3. The emergency transport dispatch system according to claim 2, wherein the disease identification means presents a possibility of a disease occurring in the patient as a score, for each of preset diseases, on the basis of results of the responses to the triage questions, and identifies a disease having the score exceeding a preset threshold thereof, as the disease having a high possibility of occurring in the patient.

4. The emergency transport dispatch system according to claim 3, wherein the driver candidate extraction means sets an extraction condition in such a manner as to extract drivers registered as available for transport for all diseases having the scores exceeding the thresholds thereof, as the driver candidates, and executes the extraction process.

5. The emergency transport dispatch system according to claim 4, wherein the driver candidate extraction means further sets an extraction condition in such a manner as to extract drivers registered as available for handling unconscious patients in a case where a response to a triage question for checking whether or not the patient is conscious indicates that the patient is unconscious, further sets an extraction condition in such a manner as to extract drivers registered as available for handling patients in cardiopulmonary arrest in a case where a response to a triage question for checking whether or not the patient is in cardiopulmonary arrest indicates that the patient is in cardiopulmonary arrest, and executes the extraction process.

6. The emergency transport dispatch system according to claim 2, wherein
the triage result acquisition means displays transport target exclusion questions used to determine whether or not to exclude the patient from the transport target, together with the questions corresponding to the triage items, obtains responses to the questions from the operator, and consequently acquires the responses to the transport target exclusion questions, and
the emergency transport dispatch system further comprises a recommendation means configured to determine that the patient is a patient who is not appropriate for emergency transport using a vehicle that is not designed specifically for emergency transport in a case where the responses to the transport target exclusion questions include a response indicating applicable to the patient, and recommend transport by himself/herself.

7. The emergency transport dispatch system according to claim 1, wherein the medical institution extraction means extracts medical institutions capable of handling the disease having a high possibility of occurring in the patient, as the medical institutions as the candidates for the medical institution to which the request for acceptance is made.

8. The emergency transport dispatch system according to claim 7, wherein in a case where there is a time limit to treatment of the disease having a high possibility of occurring in the patient, the medical institution extraction means narrows down the candidates for the medical institution to which the request for acceptance is made, considering an elapsed time from onset to the present, travel times to the medical institutions, and times from arrival of the patient at the medical institutions to start of treatment.

9. The emergency transport dispatch system according to claim 1, wherein in a case where the driver decided by the driver decision means agrees to the transport of the patient, the request information transmission means transmits the request information to the dispatch terminal operated by the driver.

10. The emergency transport dispatch system according to claim 1, wherein in a case where the driver decided as the driver to whom the request for transport is made rejects the transport of the patient, the driver decision means decides that a driver having the next shortest travel time to the current location of the patient after the driver who rejected the transport of the patient among the driver candidates extracted by the driver candidate extraction means is the driver to whom the request for transport is made.

11. The emergency transport dispatch system according to claim 1, wherein in a case where the medical worker of the medical institution decided by the medical institution decision means agrees to the acceptance of the patient, the transport information transmission means transmits the transport information to the medical staff terminal operated by the medical worker.

12. The emergency transport dispatch system according to claim 1, wherein
in a case where the medical worker of the medical institution decided as the medical institution to which the request for acceptance is made rejects the acceptance of the patient, the medical institution decision means decides that a medical institution having the next shortest travel time after the medical institution that rejected the acceptance of the patient among the medical institutions as the candidates for the medical institution to which the request for acceptance is made, which have been extracted by the medical institution extraction means, is the medical institution to which the request for acceptance is made.

13. A dispatch management apparatus for dispatching a vehicle that is not designed specifically for emergency transport for the purpose of emergency transport of a patient, the dispatch management apparatus comprising:
a triage result acquisition means configured to acquire a result of triage of a patient;
a patient current location identification means configured to identify a current location of the patient;
a disease identification means configured to identify a disease having a high possibility of occurring in the patient on the basis of the result of triage acquired by the triage result acquisition means;
a driver candidate extraction means configured to perform an extraction process for extracting drivers capable of handling emergency transport of the patient, as driver candidates, from preregistered drivers on the basis of the disease identified by the disease identification means;
a driver current location identification means configured to identify a current location of each of the drivers extracted by the driver candidate extraction means;
a driver decision means configured to decide that a driver having a short travel time to the current location of the patient among the driver candidates extracted by the driver candidate extraction means is a driver to whom a request for transport is made, on the basis of the current location of the patient identified by the patient current location identification means and the current locations of the drivers identified by the driver current location identification means; and
a request information transmission means configured to transmit information for requesting the transport of the patient to a terminal operated by the driver decided by the driver decision means, wherein
in a case where the disease identified by the disease identification means is an infectious disease, the driver candidate extraction means sets an extraction condition in such a manner as to extract drivers of vehicles registered as available for handling transport of infected persons, and performs the extraction process.

14. The dispatch management apparatus according to claim 13, wherein the triage result acquisition means displays triage questions corresponding respectively to preset triage items on a triage item by triage item basis, obtains responses to the triage questions from an operator, and consequently acquires the result of triage of the patient.

15. The dispatch management apparatus according to claim 14, wherein the disease identification means presents a possibility of a disease occurring in the patient as a score, for each of preset diseases, on the basis of results of the responses to the triage questions, and identifies a disease having the score exceeding a preset threshold thereof, as the disease having a high possibility of occurring in the patient.

16. The dispatch management apparatus according to claim 15, wherein the driver candidate extraction means sets an extraction condition in such a manner as to extract drivers registered as available for transport for all diseases having the scores exceeding the thresholds thereof, as the driver candidates, and executes the extraction process.

17. The dispatch management apparatus according to claim 16, wherein the driver candidate extraction means further sets an extraction condition in such a manner as to extract drivers registered as available for handling unconscious patients in a case where a response to a triage question for checking whether or not the patient is conscious indicates that the patient is unconscious, further sets an extraction condition in such a manner as to extract drivers registered as available for handling patients in cardiopulmonary arrest in a case where a response to a triage question for checking whether or not the patient is in cardiopulmonary arrest indicates that the patient is in cardiopulmonary arrest, and executes the extraction process.

18. The dispatch management apparatus according to claim 14, wherein
the triage result acquisition means displays transport target exclusion questions used to determine whether or not to exclude the patient from the transport target, together with the questions corresponding to the triage items, obtains responses to the questions from the operator, and consequently acquires the responses to the transport target exclusion questions, and
the dispatch management apparatus further comprises a recommendation means configured to determine that the patient is a patient who is not appropriate for emergency transport using a vehicle that is not designed specifically for emergency transport in a case where the responses to the transport target exclusion questions include a response indicating applicable to the patient, and recommend transport by himself/herself.

19. The dispatch management apparatus according to claim 13, wherein in a case where the driver decided by the driver decision means agrees to the transport of the patient, the request information transmission means transmits the request information to the terminal operated by the driver.

20. The dispatch management apparatus according to claim 13, wherein in a case where the driver decided as the driver to whom the request for transport is made rejects the transport of the patient, the driver decision means decides that a driver having the next shortest travel time to the current location of the patient after the driver who rejected the transport of the patient among the driver candidates extracted by the driver candidate extraction means is the driver to whom the request for transport is made.
